(19) United States
(12) Patent Application Publication
(10) Pub. No.: US 2026/0256541 A1
(43) Pub. Date: Sep. 3, 2026

(54) LASER-GUIDANCE ROBOT FOR VISUALLY PROJECTING A GUIDE TO A SURGERY PLAN, PROJECTION METHOD, AND LASER-GUIDANCE ROBOT SYSTEM

(71) Applicant: B. Braun New Ventures GmbH, Freiburg im Breisgau (DE)

(72) Inventors: Amir Sarvestani, Freiburg (DE); Tim Beyl, Baden-Baden (DE); Robert Jakob, Ihringen (DE)

(21) Appl. No.: 18/878,504

(22) PCT Filed: Jun. 19, 2023
(86) PCT No.: PCT/EP2023/066472

§ 371 Date: Dec. 23, 2024

(30) Foreign Application Priority Data
Jun. 24, 2022 (DE) 10 2022 115 822.5

(51) Int. Cl. A61B 90/13; A61B 34/10; A61B 34/20; A61B 34/32; A61B 90/00

(52) U.S. Cl. CPC A61B 90/13; A61B 34/32; A61B 2034/107; A61B 2034/2055; A61B 2034/2065; A61B 2034/2072; A61B 2090/366; A61B 2090/373

(57) ABSTRACT

A laser-guidance robot for visually projecting a surgical guide onto a surgery region of a patient includes a guidance robot having a robot arm and a robot head. A tracking system can detect a position and/or an orientation of the robot head and of the patient. The robot head has a projection laser on the robot head. The position and orientation of the projection laser can be adjusted by adjusting the robot head. A control unit determines a target position and target orientation of the projection laser relative to the surgery region of the patient and controls the robot arm such that the projection laser is moved into the target position and target orientation. At least one guide axis is visually displayed by the projection laser projecting the laser radiation in the target orientation. The laser-guidance robot can be part of a laser-guidance robot system and be used in a projection method.

### CROSS-REFERENCE TO RELATED APPLICATIONS

This application is the United States national stage entry of International Application No. PCT/EP2023/066472, filed on Jun. 19, 2023, and claims priority to German Application No. 10 2022 115 822.5, filed on Jun. 24, 2022. The contents of International Application No. PCT/EP2023/066472 and German Application No. 10 2022 115 822.5 are incorporated by reference herein in their entireties.

### FIELD

The present disclosure relates to a laser-guidance robot for visually projecting a guide to a surgery plan onto an intervention region of a patient. In addition, the disclosure relates to a projecting method for visually projecting a guide and a laser-guidance robot system comprising the laser-guidance robot and a medical instrument or product.

### BACKGROUND

Navigation systems and/or operation robots are used in computer-assisted surgeries. When navigation systems are used, surgical instruments are detected and followed/tracked. In particular, a position and an orientation of the surgical instruments in space are detected. The detected information is correlated and combined with preoperative images that were detected before the surgery. These preoperative images are, in particular, a model of a patient or a patient's body. This digital model is created, for example, by a computed tomography image (CT image) and/or a magnetic resonance imaging image (MRI image). When using operation robots, surgical instruments are moved to a target position by one or more robot arms, wherein the target position is determined on the basis of the preoperative images.

Both the use of navigation systems and operation robots have certain disadvantages. The use of navigation systems imposes restrictions in terms of freedom of movement due to the attachment of navigation trackers to surgical instruments. The use of operation robots, on the other hand, reduces the working space or working area due to the need for an end effector and is also more complicated in terms of the system.

For this reason, solutions have been developed in which the surgery is performed conventionally by a surgeon, but the surgeon is supported by a projection of a preoperatively calculated surgery plan.

DE 10 2017 127 791 A1 discloses a medical control system. A projector, configured for example in the form of a laser, projects a surgical instruction onto a patient. The surgical instruction may represent an intervention location on the patient as well as a position of vulnerable tissue or organs on the patient. The projected surgical instruction supports an attending surgeon, since the projection can be used for orientation. The surgical instruction was determined before surgery by scanning the patient.

EP 3 733 111 A1 describes a laser-based system for providing instructions during surgeries. The system has a semi-circular arm (C-arm X-ray device) with a laser projector. The (X-ray) arm scans the body of a patient and displays the scan on a screen. A user can edit operation information directly on the screen. For example, the user can enter operation information via a touch screen. A control unit converts the operation information and the laser projector projects the operation information onto the patient's body. However, the system has the voluminous and bulky C-shaped arm, which has a negative impact on handling during surgery. Furthermore, the C-shaped arm cannot be moved at will during surgery and requires strategic repositioning.

There are also (robotic) systems known that use high-energy lasers to cut bone. A surgery plan is created based on preoperative data. An effector is controlled by an optical navigation system and cuts bones using lasers based on the surgery plan. This is advantageous in that the robot produces a very clean cut edge and laser cutting does not produce any chips. However, laser cutting cannot be used for biopsies or other surgical interventions such as the placement of screws or similar items.

Furthermore, a system with several stationary lasers is known from ‘Liao, et. al—Precision-guided surgical navigation systems using laser guidance and 3D autostereoscopic image overlay’. At least two lasers are arranged at a distance. Both lasers project a laser beam onto a surgery surface. The intersection line of the two lasers represents a surgery line. For example, the intervention line can be displayed during a surgery. However, the stationary system lacks the flexibility for a number of surgeries.

In summary, the systems known from the prior art can indicate to the surgeon at which location he/she has to perform an intervention and can also mark relevant anatomical orientation points such as organs that the surgeon must not injure during surgery. However, the information is only projected onto the patient from above. As a result, only a two-dimensional image is displayed without providing any spatial information.

### SUMMARY

Therefore, the objects of the present disclosure are to overcome or at least reduce the disadvantages of the prior art and in particular to provide an effective, flexible laser-guidance robot and a projecting method and a laser-guidance robot system which provides a user, in particular an attending surgeon, with further spatial information for guidance and navigation and in particular enables a function of a display at which location and in particular at which angle he/she can perform an intervention. A further partial object can be seen in providing a low-cost and movable laser-guidance robot and guidance robot system, which can be used in particular in a movable manner and has as little impact as possible on an operating room in terms of the volume required. In particular, an intervention region still has to be freely accessible for the surgeon.

The core idea of the present disclosure is therefore not only to be able to display a location by combining a robot as a positioning system and a laser as a display system with a laser radiation having a longitudinal axis, but also to be able to visually display an axis in space to this location for the surgeon, so that the surgeon can use the information of the axis to align medical products or instruments accordingly. The decisive factor here is that the robot can steer and thus move the laser into a predeterminable position and orientation relative to the patient and thus to the intervention location, the laser aims at a target point of the intervention region in this pose and aligns a laser radiation with a longitudinal axis and thus a guiding axis to this target point to be aimed at and displays it visually. This enables the surgeon not only to position the medical products, such as a biopsy needle, or instruments, such as a screwdriver, at an indicated position, but also to align a longitudinal axis according to the indicated guiding axis.

In other words, the present disclosure relates to a laser-guidance robot for visually projecting a (surgical) guide, in particular a preoperatively calculated surgery plan, onto an intervention region of a patient. The laser-guidance robot comprises a (guidance) robot with a robot arm movably articulated or connected to a robot base and a terminal/distal robot head/end portion connected to the robot arm, and further comprises a tracking system, in particular an optical navigation system or a robot kinematics-based tracking system (where the kinematics of the (guidance) robot is used to preferably determine the position and orientation of the laser, in particular relative to the patient), which is adapted to detect a position and/or an orientation of the robot head and/or a position and/or an orientation of the patient and thus of the intervention region. The laser-guidance robot further comprises a projection laser whose emitted laser radiation is preferably within a range visible to the human eye, and which is arranged, in particular attached, to the robot head, so that a position and orientation of the projection laser can be both detected and adjusted indirectly via the robot head.

The laser-guidance robot furthermore has a control unit which is adapted to determine a target position and target orientation of the projection laser in relation to the intervention region of the patient and to control the robot arm with the robot head and the projection laser in such a way that the projection laser is moved/travels to the target position and target orientation and visually displays at least one guiding axis on the intervention region via its (visual) projection of the laser radiation in the target orientation.

In particular, the control unit calculates the surgery plan and thus at least one predefined intervention location for the intervention with an associated target position and target orientation of the projection laser from preoperative images. Furthermore, the control unit may in particular be adapted to calculate an intervention axis which is located at the intervention location at a calculated intervention angle. The robot arm is movable and has a number of degrees of freedom. The projection laser is arranged/attached to a distal end portion of the robot arm or of the robot head and can therefore be moved with the robot arm and can be controlled by the robot. The tracking system detects the position and orientation of the robot head and thus (in particular via a static, known or predeterminable transformation from the coordinate system of the robot head to the projection laser) the position and orientation of the projection laser in space. In this way, reaching the target position and target orientation of the projection laser can also be both driven and detected. The degrees of freedom of the robot arm are used to arrange/position and align the projection laser in such a way that the emitted laser radiation of the projection laser (i.e. the longitudinal axis of the laser radiation) corresponds to the calculated intervention axis with the calculated intervention angle. The laser radiation emitted by the projection laser, which is directly visually visible since it emits wavelengths in the visible range, or is in a non-visible range but causes fluorescence in instruments, thus forms the guiding axis for the surgery or intervention.

The target position and the target orientation of the projection laser are the position and the orientation of the projection laser in which the guiding axis projected by the laser radiation corresponds to the calculated intervention axis and in particular is aligned with a target point to be aimed at in an intervention region.

Preoperative images are acquired prior to surgery, preferably by computed tomography (CT image) and/or magnetic resonance imaging (MRI image).

The term “tracking system” describes a technical system that enables spatial localization and makes it possible to detect the position and/or orientation of a target object.

The term “position” refers to a geometric position in three-dimensional space, which in particular is specified using coordinates of a Cartesian coordinate system. In particular, the position can be specified by the three coordinates X, Y and Z.

The term “orientation” in turn indicates an alignment (such as position) in space. It can also be said that the orientation indicates an alignment with direction or rotation in three-dimensional space. In particular, the orientation can be specified using three angles.

The term “pose” covers both a position and an orientation. In particular, the pose can be specified using six coordinates, three position coordinates X, Y and Z and three angular coordinates for the orientation.

In summary, in other words, the core of the present disclosure can be seen in that a projection laser is positioned and aligned by a robot arm in such a way that the laser radiation emitted by the projection laser corresponds to an intervention axis to be approached, in particular a preoperatively calculated intervention axis for a surgery. The surgeon is given the option of not only visually displaying the position in the intervention region, but also an axis (with a corresponding intervention angle).

The laser-guidance robot according to the disclosure enables a user or medical professional, in particular a surgeon, to follow the calculated intervention axis as precisely as possible. Thus, the advantages of an operation robot, i.e. the exact tracking of a preoperatively calculated operation trajectory, can be combined with the high flexibility and easy handling of a (conventional) manual surgery. In particular, the treating surgeon is shown the angle at which he/she should perform an intervention. For example, the projected guiding axis may indicate how a biopsy needle is to be inserted into the patient's body or at what angle a pedicle screw is to be screwed into the patient's spine. By supporting the surgeon through the display/projection of the guiding axis, surgery can be carried out precisely and safety for the patient is increased. Surgery-related trauma is reduced.

The object of the present disclosure is further solved by a projecting method according to the disclosure for projecting a guide (with guiding axis), in particular a (preoperatively calculated) surgery plan, with the projection laser attached to the robot head. The projecting method has the following steps. A tracking system detects a position and/or an orientation of the robot head and thus (via a determinable transformation between the robot head and the projection laser) the pose of the projection laser in space. The control unit calculates a guiding axis, in particular based on preoperative images. The control unit furthermore calculates the target position and the target orientation of the projection laser based on the calculated intervention axis, in particular from the preoperative images. The robot arm then moves the robot head with the projection laser to the calculated and predetermined target position and target orientation. The projection laser projects the laser radiation in the target orientation in such a way that at least one guiding axis is visually displayed on the intervention region. In particular, the projected guiding axis corresponds to the calculated intervention axis.

The surgery plan is preferably calculated by the control unit based on the preoperatively detected images. The calculated surgery plan has at least one intervention location (target point) as well as the intervention axis and/or the intervention angle. In particular, the surgery plan has a rectilinear trajectory.

Using the projecting method according to the disclosure, the calculated intervention axis of the planned surgery may be displayed as the projected guiding axis. This allows the surgeon to operate with high precision by following the guiding axis and thus the intervention axis.

Advantageous further embodiments of the present disclosure are explained in particular below.

Preferably, the control unit calculates at least a first target position and target orientation of the projection laser on the basis of the surgery plan stored in a memory unit. The control unit may control the robot arm in such a way that the projection laser projects its laser radiation onto the calculated intervention location in the target position and target orientation and visually displays the guiding axis with the calculated target orientation. The laser radiation and thus the guiding axis thus show the intervention location calculated by the control unit.

The memory unit may be configured as part of the control unit or as part of an external control unit.

It may be advantageous for the control unit to drive, i.e. position and orient the projection laser relative to a predefined intervention location via the robot arm in such a way that the guiding axis is at a predetermined angle to the predefined intervention location (or target point of the intervention location). As a result, the laser radiation emitted by the projection laser indicates the intervention angle. In particular, the intervention angle is the angle calculated as the optimal angle for the surgical intervention. The intervention angle can be calculated based on the preoperative data set.

According to a further optional aspect of the present disclosure, the surgery plan comprises a, preferably rectilinear, (operation) trajectory, (intermediate) target points (in particular a target point on the rectilinear operation trajectory, for example to indicate an axis and a target position for an instrument) and/or outlines of a (operation) target. The surgery plan also preferably comprises at least one intervention location, an intervention axis and/or an intervention angle. The control unit may set the target orientation (with associated target position) of the projection laser in such a way that the guiding axis corresponds to the intervention axis. As a result, the calculated intervention axis is displayed to the user by the projected guiding axis. The guiding axis may therefore correspond to (any) intervention axis because the projection laser can be moved to almost any position and orientation by the (movable) robot arm. During surgery, the surgeon only has to follow the projected guiding axis in order to position his/her (medical) instrument or product at the correct intervention angle. This increases the probability that the surgeon will meet the calculated intervention axis.

Preferably, the laser radiation emitted by the projection laser is adapted in such a way that it visually indicates or points to a focal point at a predetermined intervention depth. The intervention depth may be the subject matter of the calculated surgery plan and can be calculated by the control unit based on the preoperative images. If the projection laser displays the calculated intervention depth during surgery, the likelihood of the surgeon inadvertently intervening at the wrong depth is minimized. Complications and trauma can thus be avoided. The surgery is gentler on the patient and recovery times after surgery are shortened.

It may be advantageous for the laser radiation projected by the projection laser to form a conical structure with a focal point, wherein the focal point indicates the intervention depth. The intervention depth is visually represented by the focal point at the tip of the conical structure, which is turned away from the projection laser, so that rings or oval shapes are formed in an intervention region above the intervention depth (i.e. facing the projection laser), which converge on the focal point at the intervention depth. This means that the emitted laser radiation is configured in a circular cross-section (with a changing diameter) and is not point-shaped. The laser radiations intersect each other at an intersection point, which forms the tip of the cone. The intersection point indicates the optimal (calculated) intervention depth. If the surgeon has not yet reached the desired intervention depth, the laser radiation is displayed as rings on a projection surface. The size of the rings may indicate how far away the surgeon is from the intervention depth. The larger the projected rings, the further the distance from the desired intervention depth. If the surgeon follows the intervention axis, the laser radiation may be configured as concentric rings. If the surgeon deviates from the intervention axis during the intervention, i.e. the intervention direction is at an angle to the laser radiation, and a projection surface on the patient is not orthogonal to the longitudinal axis, the laser radiation may be displayed as an oval shape. This indicates to the surgeon that the intervention direction does not match the calculated intervention axis.

Preferably, the medical instrument or product may have a predetermined projection area or device where the user can see the projection of the projection laser.

In particular, the laser radiation projected by the projection laser forms a hyperboloid/double cone structure (such as an hourglass), with a focal point for a visual representation of the intervention depth at the common tip of the double cone structure, so that in an intervention region, rings or oval shapes form above the intervention depth, converging to the focal point at the intervention depth and diverging again as rings or oval shapes below the intervention depth. If the surgeon has penetrated too deeply during the intervention, the laser radiation may appear as rings that increase in size as the distance to the desired intervention depth increases.

Preferably, the projection laser emits at least two straight laser beams, which have an angle to each other and intersect each other and the intersection point of the at least two laser beams indicates the calculated intervention depth. As explained above, the laser radiation of the projection laser cannot be emitted parallel or converging. As a result, the (straight, non-fanning) multiple emitted laser beams intersect and form the intersection point. The calculated intervention depth is indicated by the intersection point. Two points can in particular be projected through the two straight laser beams, the distance between which decreases toward the focal point and merges into a single superimposed point.

According to a further optional aspect of the present disclosure, the laser-guidance robot comprises an input and output unit, in particular a (touch) display, for inputting control commands and/or outputting information to a medical professional, in particular a surgeon. For example, the user may select a desired end position and end orientation of the robot head and/or a target position and target orientation of the projection laser via the input and output unit and the robot moves the projection laser accordingly. The user, in particular the medical professional or the surgeon, may also enter a trajectory of the surgery via the input and output unit.

Preferably, the tracking system comprises an optical navigation system with a navigation camera and with one or more optical trackers. The trackers are provided and configured to be attached to the distal end portion or to the robot head of the robot arm and/or to the patient, so that their position and orientation in space are detected by a navigation camera of the navigation system in order to move the projection laser to the calculated target position and target orientation relative to the detected position and/or orientation of the patient and thus to the intervention region. In particular, the robot head has a rigid body with reflective markers as a robot tracker. For example, a tracker is arranged on the patient and a tracker on the robot head. The navigation camera is preferably a stereo camera that can detect the position and orientation of the tracker. In this way, the position and orientation of the patient and thus of the intervention region and the position and orientation of the robot head and thus of the projection laser can be detected. In particular, a registration of the patient may be performed based on the patient tracker or the control unit may be adapted to perform a registration of the patient via the tracking system. In particular, an initial position of the robot head may be detected by the optical navigation system and the robot head may be moved to the target position relative to the patient's intervention region and may be aligned in the target orientation. The optical navigation system recognizes when the target position and the target orientation have been reached.

Preferably, the position and orientation of the robot head may be detected via servomotors of the guidance robot (via corresponding robot kinematics). This means that no optical tracking or navigation system with the camera and trackers would be required to detect the position and orientation of the robot head. The precisely controllable servomotors are already present in the guidance robot.

Furthermore, the projection laser may preferably be moved relative to the robot head by servomotors in order to actively adjust the orientation of the projection laser relative to the robot head. This means that the projection laser also has at least one degree of freedom in relation to the robot head. This would make the alignment of the projection of the laser radiation by the projection laser even more flexible.

According to another optional aspect of the present disclosure, preoperative images of the patient are stored in a memory unit/data provision unit, in particular in the form of computed tomography and/or magnetic resonance imaging (CT-images and/or MRI-images). Based on a registered patient, the control unit calculates an intervention axis included in the preoperative images and a best possible associated target position and target orientation and the projection laser is moved/displaced accordingly so that the guiding axis is displayed in the best possible way and in particular corresponds to the intervention axis, i.e. is aligned coaxially to it.

Preferably, the guidance robot has at least one projection laser, preferably two projection lasers, and the projection laser or lasers are adapted to emit at least two different wavelengths in order to display two different colors and, in particular, to color-code different targets in the intervention region. The different colors of the laser may be used to indicate different relevant orientation points such as organs or different targets such as a biopsy and/or a tumor or a medical product.

According to a further optional aspect of the present disclosure, the projection laser may also be brought into the calculated end position by manually moving the robot arm, while the (optical) navigation system detects the current position and/or orientation of the projection laser during the manual movement and indicates that the calculated target position and/or target orientation has been reached. This means that the robot arm can also be positioned manually by the surgeon and the surgeon can see via the display device approximately how far away it is from its target position and target orientation.

In particular, the target position may be set on a longitudinal axis of the laser radiation and may be displayed in particular. In other words, the guiding axis may be aligned in space, but can be moved along the longitudinal axis in the case of a parallel laser radiation, while continuing to display the guiding axis.

In particular, the projection laser may be adapted to adapt the laser radiation in the form of intersecting laser beams so that a distance between the projection laser and the intersecting laser beams is changed to set the focal point. In particular, the control unit may be adapted to maintain the focal point during a translational displacement of the robot head with the projection laser along the longitudinal axis of the laser radiation by changing the distance between the projection laser and the intersecting laser beams accordingly.

The object of the present disclosure is further solved by a laser-guidance robot system comprising a laser-guidance robot according to the present disclosure, in particular according to one of the aspects explained above and comprising a medical, in particular surgical, instrument, in particular a screwdriver, and/or a medical product, in particular a biopsy needle or a pedicle screw. The laser-guidance robot projects the guiding axis onto an intervention region via the projection laser. The medical instrument or product may be aligned with a longitudinal axis along the guiding axis in that a, preferably proximal, portion of the instrument or product (along the longitudinal axis) has a projection surface onto which the projection laser projects the laser radiation when the instrument or product is aligned with its longitudinal axis substantially parallel, in particular coaxial, to the guiding axis.

Preferably, the medical device has an optical marking at a proximal end that indicates whether the surgical device is aligned in the intervention axis. The proximal marking may be concentric circles, for example. If the laser radiation falls on the proximal marker in such a way that the dot-shaped projection is in the center of the marker or a ring-shaped projection is concentric with the circles of the marker, the surgical product is aligned in the guiding axis or the intervention axis. The other rings may indicate a certain angle, which helps the surgeon to decide whether the selected intervention axis of the instrument or product is still within a tolerance range to the optimal intervention axis. In particular, a target or target-like rings may be applied to the projection surface so that the surgeon is shown whether he is coaxial with the guiding axis when the laser dot lights up in the center of the target.

The laser-guidance robot according to the disclosure may be modified in particular according to at least one of the following embodiments: The robot head may comprise a surgical microscope; The laser-guidance robot may be combined with a known navigation system; The laser-guidance robot may be combined with a robotic surgical system; The projection laser may be integrated into an optic of the surgical microscope; The projection laser may be attached to the robot head or configured to be attachable to the robot head; The laser-guidance robot may be used as a robotic laser pointer controlled by an external user; The navigation system may be based on an infrared system with an infrared camera and/or may be based on electromagnetic tracking (EM tracking) and/or may be based on a computer vision system.

The laser-guidance robot and the projecting method may be used as follows. The following applications are examples. The list is not exhaustive. A planned intervention location/incision site may be projected onto the patient's skin. A path or a trajectory (in particular a straight line) from the intervention location to a tumor may be projected. Critical structures such as vessels may be marked to avoid unintentional damage. The outer borders/perimeters of a tumor may be marked. A trajectory of a biopsy may be marked and displayed via the guiding axis. A trajectory of a (planned) bone screw may be marked via the guiding axis. The laser may be used as a remote-controlled laser pointer with which an assisting surgeon may point to anatomical orientation points to guide a performing or operating surgeon.

The objects are solved with respect to a computer-readable storage medium and with respect to a computer program, respectively, in that the latter comprises instructions which, when executed by a computer, cause the computer to perform the steps of the projecting method according to the present disclosure.

### BRIEF DESCRIPTION OF THE DRAWINGS

The present disclosure is explained in more detail below with reference to preferred embodiments with the aid of Figures.

FIG. 1 shows a laser-guidance robot according to a preferred embodiment of the present disclosure;

FIG. 2 shows a schematic representation of a laser-guidance robot according to a further embodiment of the present disclosure;

FIG. 3 shows a partial section of the laser-guidance robot with a robot head with a projection laser;

FIG. 4 shows a schematic representation of a display view with an intervention axis in a tissue;

FIG. 5 shows a system according to a preferred embodiment of the present disclosure comprising a medical product in the form of an instrument and the robot head;

FIG. 6a shows a schematic view of an overlapping laser radiation of the projection laser, resulting in a double-cone projection;

FIG. 6b shows a schematic view of a simple, straight laser radiation of the projection laser;

FIG. 6c shows a schematic view of a parallel laser radiation of the projection laser to create a hollow cylindrical projection; and

FIG. 7 shows a flow diagram of a projecting method according to a preferred embodiment of the present disclosure.

The Figures are merely schematic in nature and are intended only to aid understanding of the disclosure. Identical elements are provided with the same reference signs. The features of the various embodiments can be interchanged.

### DETAILED DESCRIPTION

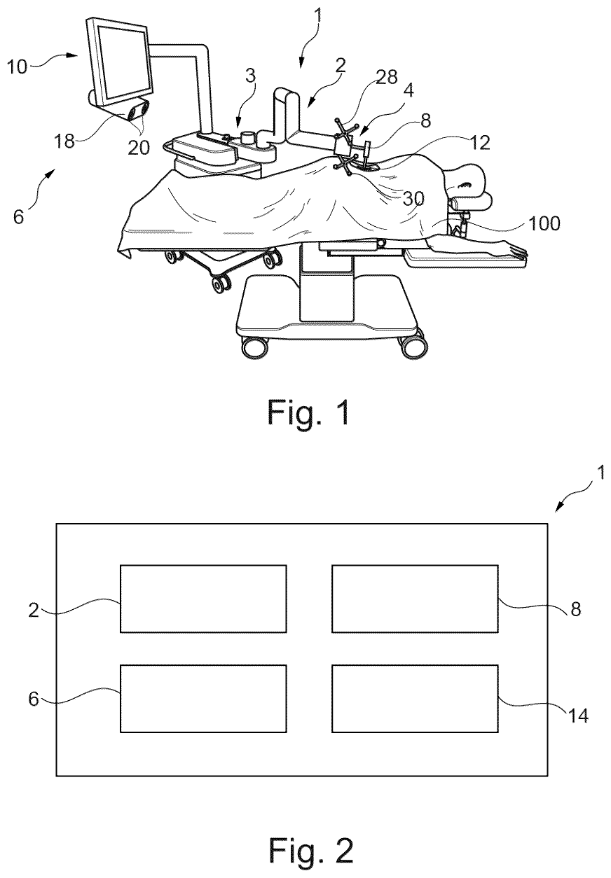

FIG. 1 shows a laser-guidance robot 1 positioned next to a patient 100. The laser-guidance robot 1 has a guidance robot with a (movable) robot arm 2, which is movably articulated or attached to a robot base 3. The robot arm 2 has a distal robot head 4 connected to the robot arm 2. The laser-guidance robot 1 furthermore has a tracking system 6, a projection laser 8 and, in this embodiment, an input and output unit 10. The tracking system 6 detects a position and an orientation of the robot head 4 as well as a position and an orientation of the patient 100 and thus of the intervention region. The projection laser 8 is attached to the robot head 4 so that the position and orientation of the projection laser 8 can be adjusted indirectly via the robot head 4. The projection laser 8 emits laser radiation 12, which in this embodiment lies in a range visible to the human eye between 380 nm and 750 nm.

The laser-guidance robot 1 furthermore has a control unit 14 (shown in FIG. 2), which is adapted to determine a target position and target orientation of the projection laser 8 in relation to the intervention region of the patient 100. The control unit 14 controls the robot arm 2 with the robot head 4 and the projection laser 8 in such a way that the projection laser 8 is moved/displaced into the target position and target orientation. At least one guiding axis 16 and also an intervention location (target point) are visually displayed on the intervention region via a (visual) projection of the laser radiation 12 of the projection laser 8 in the target orientation. In this way, in contrast to the known prior art, the surgeon is not only provided with a projection of an intervention point on the patient, but a guiding axis is also visually displayed.

The robot base 3 of the laser-guidance robot 1 is preferably arranged on a rollable or movable cart in order to provide a mobile laser-guidance robot 1 that can be moved to different positions in an operating room. The cart houses the control unit 14 (not shown in FIG. 1, but shown in FIG. 2), a power supply (not shown) and optionally a battery (not shown) for emergency power supply. The cart furthermore houses a memory unit 17 (not shown).

The robot arm 2 has several robot arm segments and joints so that the robot arm 2 is movable in various translational degrees of freedom and at least one rotational degree of freedom. The robot head 4 is hinged to a distal end of the robot arm 2 so that it is rotatable about an axis. Thus, the robot head 4 and therefore the projection laser 8 is translationally movable at least in space and can be rotated about a longitudinal axis of the distal end of the robot arm 2 in order to set a target position in space (relative to the patient) and a target orientation with a corresponding guiding axis.

FIG. 2 shows a schematic representation of the laser-guidance robot 1 with the robot arm 2 together with the robot head 4, the tracking system 6, the projection laser 8 and the control unit 14. The tracking system 6 has an optical navigation system 18 with a navigation camera 19 with at least two spaced-apart camera lenses 20 and a number of (optical) trackers 22 (markers). The trackers 22 each have three or four arms 24 which project in different directions from a common point. The respective arms 24 are preferably arranged perpendicularly or at an angle of 120° to each other. A tracker sphere 26 is arranged at the outer end of each arm. The tracker spheres 26 are configured in such a way that they can be easily detected by the navigation camera 19 and can also emit light signals. The respective tracker spheres 26 of a tracker 22 lie in a common plane. By aligning the plane in space, the navigation system 8 can calculate how the respective tracker 22 is arranged on an object. Such trackers 22 are known.

A laser tracker 28 is attached to the robot head 4 at the distal end of the robot arm 2. This allows the position and orientation of the robot head 4 in space to be detected. A patient tracker 30 is arranged on the patient 100 in order to detect the position and orientation of the patient 100 in space and to use this information to register the patient.

The input and output unit 10 is a touchscreen display via which information can be output to a user and information can be entered into the laser-guidance robot 1 by the user.

The control unit 14 of the laser-guidance robot 1 can determine the target position and the target orientation of the projection laser 8 in relation to the patient's intervention region and can control the robot arm 2. In particular, the control unit 14 can control the robot arm 2 in such a way that the projection laser 8 is moved/displaced to the target position and target orientation. The control unit 14 calculates the target position and target orientation and an intervention axis 32 from preoperative images of a patient. The preoperative images are acquired by computer tomography and/or magnetic resonance imaging of the patient and are provided to the control unit 14 by a data provision unit. The control unit 14 displaces the projection laser 8 accordingly so that the guiding axis 16 corresponds to the intervention axis 32.

For example, the robot head 4 has an (intervention) camera 34 through which the user can inspect an intervention or surgeries site. The projection laser 8, which emits the laser radiation 12, is arranged on the robot head 4. The laser radiation 12 emitted by the projection laser 8 displays the guiding axis 16 at the intervention region or a surgery plan. The surgery plan may, for example, be the intervention location at which the surgeon is to make the incision. The surgery plan may also be a trajectory from the incision site to a target such as a biopsy area, a tumor or a (surgery) screw, and the trajectory may be visualized at least in sections via corresponding guiding axes.

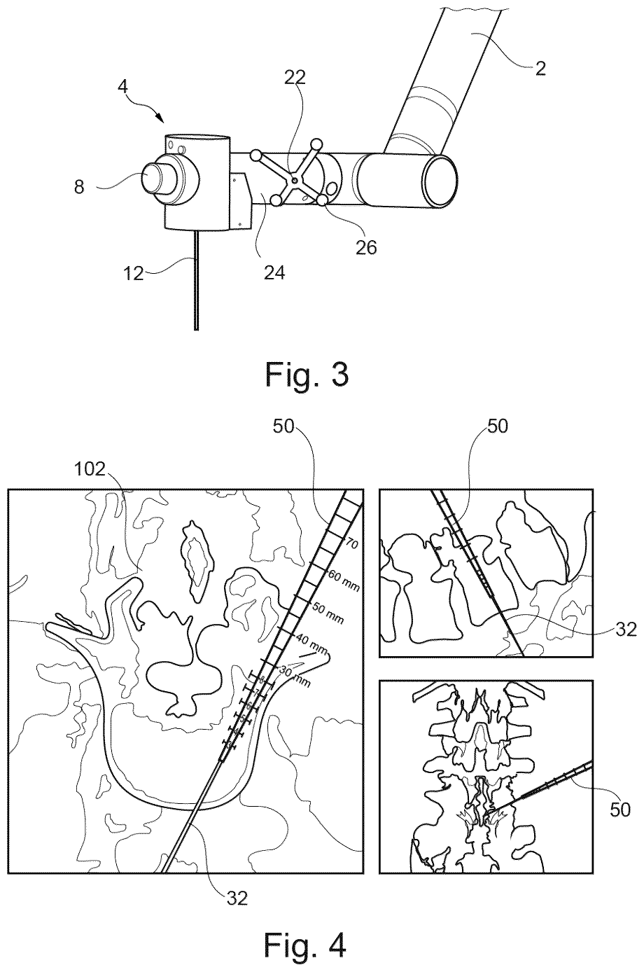

FIG. 3 shows the distal end of the robot arm 2 with the robot head 4, the tracker 22 and the projection laser 8. The tracker 22, which is the laser tracker in particular, enables the (optical) navigation system 18 to detect the position and orientation of the robot head 4 in space in real time. This enables the control unit 14 to recognize when the robot head 4 has arrived at the target position and target orientation.

FIG. 4 shows the calculated intervention axis 32 in a preoperative image of the patient 100. The body or a tissue 102 of the patient 100 is detected or scanned before the surgery. The control unit 14 or an external computing unit (not shown) uses these preoperative images to calculate the surgery plan with at least one operation trajectory 50. The intervention axis 32 is also calculated and, in the present case, a target point on the intervention axis of the operation trajectory is also calculated with corresponding distances. The aim of the surgery is to ensure that a medical instrument or product 36 follows the calculated intervention axis 32 as precisely as possible and is correspondingly stopped at the target depth in order to perform the corresponding manipulations. For this purpose, the calculated intervention axis 32 has to be made visible so that the surgeon can follow the intervention axis 32 with the medical product 36. This is realized by the present disclosure. In particular, the target point can be visualized at the target depth via intersecting laser beams.

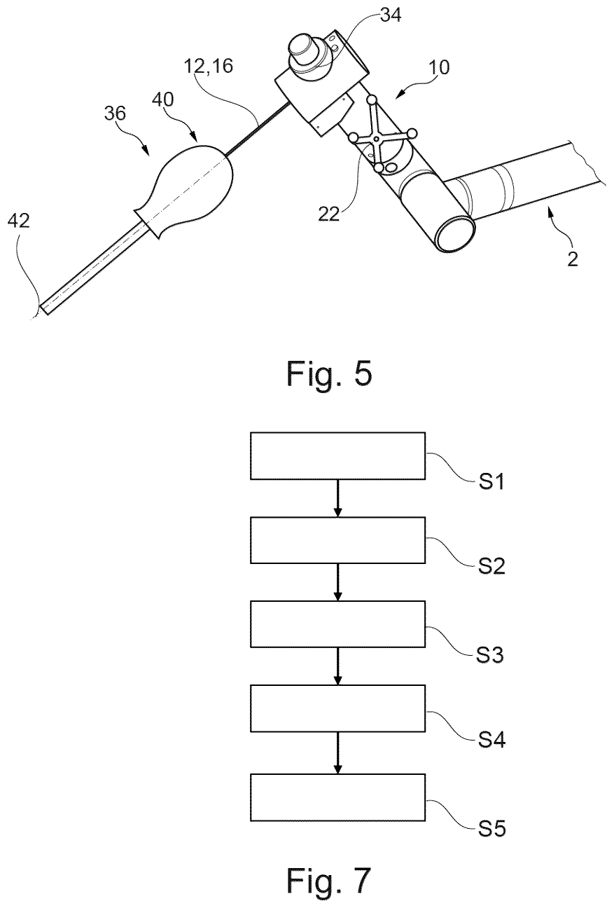

FIG. 5 shows a laser-guidance robot system of the present disclosure with the laser-guidance robot 1 and the medical product or instrument 36. The projection laser 8 emits the laser radiation 12, which represents the guiding axis 16. A longitudinal axis of the medical product 36 is aligned along the projected guiding axis 16. If the longitudinal axis of the medical product 36 corresponds to the guiding axis 16, the medical product 36 is also aligned along the calculated intervention axis 32. The degrees of freedom of the robot arm 2 allow the projection laser 8 to be aligned and positioned in such a way that the emitted laser beams 12 correspond to the projected guiding axis 16. The tracker 22 detects the positioning and/or orientation of the robot head 4 with the projection laser 8 so that the target position and target orientation can be precisely controlled. A proximal portion 40 of the medical product 36 has a marking in the form of concentric rings, which indicates whether the medical product 36 is aligned with a longitudinal axis 42 in the guiding axis 16. If a straight laser beam of the projection laser is displayed in the center of the concentric rings, a coaxial alignment of intervention axis 32 and guiding axis 16 can be assumed.

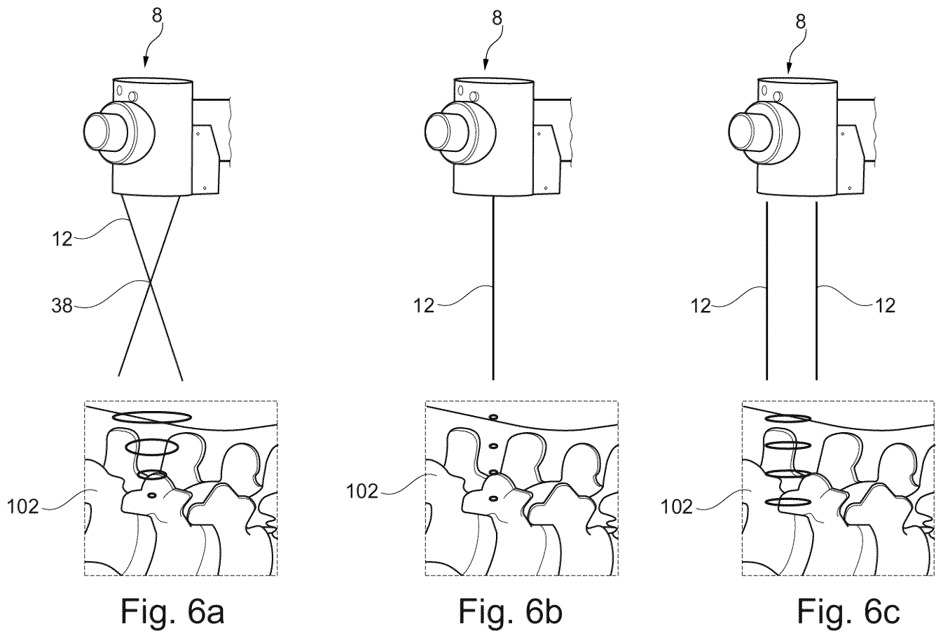

FIGS. 6a to 6c show different embodiments of a projection laser 8 with a differently configured laser radiation, each of which can be used in a laser-guidance robot 1 or laser-guidance robot system of the present disclosure.

FIG. 6a shows a projection laser 8 according to a first embodiment. Here, laser beams 12 are emitted by the projection laser 8 in the form of a double cone. The ring-shaped laser beams 12 are at an angle to each other and therefore overlap at an intersection point 38. The intersection point 38 of the laser beams 12, which form the double cone contour, can be used to display a calculated intervention depth. More precisely, the laser beams 12 converge in the shape of a cone. The tip of the cone marks the calculated/desired intervention depth. As long as the intervention depth has not been reached, this projection indicates rings or oval shapes in the intervention. When the intervention depth is reached, only a point is visible. If the incision penetrates deeper than the calculated intervention depth, the rings become larger again as the distance to the intervention depth increases.

FIG. 6b shows a projection laser 8 according to a second embodiment. The projection laser 8 is configured as a point laser or as a line laser. If the projection laser 8 is configured as a line laser, the projection laser 8 has a special lens (not shown) that fans out a point laser into a line laser (i.e. a constant cross-sectional contour along the longitudinal axis of the laser radiation as lines). In this embodiment, the laser radiation 12 cannot display a calculated or desired depth of the cut. However, the laser radiation 12 can display the guiding axis 16 very accurately.

FIG. 6c shows a projection laser 8 according to a third embodiment. Here, the hollow cylindrical laser beams 12 mark a predetermined area using circles. The marked area can be used, for example, to identify a tumor to be removed.

FIG. 7 shows a flowchart of a projecting method according to the disclosure for visually projecting a guide of a surgery plan onto an intervention region of a patient 100 with the projection laser 8, which is attached to the robot head 4 of the robot arm 2.

In a first step S1, the tracking system 6 detects the position and orientation of the robot head 4 and of the projection laser 8 in space.

In a step S2, the control unit 14 plans/calculates the intervention axis 32 based on preoperative images.

In step S3, the control unit 14 calculates the target position and target orientation of the projection laser 8.

In step S4, the robot arm 2 moves the robot head 4 with the projection laser 8 to the predetermined target position and target orientation.

In step S5, the projection laser 8 projects in its target orientation laser radiation 12 such that the guiding axis 16, which is projected by the projection laser 8, corresponds to the intervention axis 32.

In this way, the projecting method can display an axis to the surgeon.

### LIST OF REFERENCE SIGNS

1 projection device
2 robot arm
3 robot base
4 robot head
6 tracking system
8 projection laser
10 input and output unit
12 laser radiation
14 control unit
16 guiding axis
18 navigation system
19 navigation camera
20 camera lens
22 tracker
24 arm
26 tracker sphere
28 laser tracker
30 patient tracker
32 intervention axis
34 Intervention camera
36 medical product
38 intersection point
40 proximal portion
42 longitudinal axis
50 operation trajectory
100 patient
102 tissue

1.-15. (canceled)

16. A laser-guidance robot for visually projecting a guiding axis onto an intervention region of a patient, the laser-guidance robot comprising:
a guidance robot with a robot arm movably articulated to a robot base and a robot head connected to the robot arm;
a tracking system adapted to detect a position and/or an orientation of the robot head;
a projection laser configured to emit laser radiation, the projection laser arranged so that a position and orientation of the projection laser is adjustable indirectly via the robot head; and
a control unit adapted to determine a target position and target orientation of the projection laser in relation to the intervention region of the patient and to control the robot arm with the robot head and the projection laser in such a way that the projection laser is moved to the target position and target orientation and visually displays at least one guiding axis on the intervention region via projection of the laser radiation in the target orientation.

17. The laser-guidance robot according to claim 16, wherein the tracking system comprises an optical navigation system.

18. The laser-guidance robot according to claim 16, wherein the tracking system is adapted to detect a position and/or an orientation of the patient and thus of the intervention region.

19. The laser-guidance robot according to claim 16, wherein the control unit is adapted to calculate at least a first target position and first target orientation of the projection laser based on a surgery plan stored in a memory unit and to control the robot arm so that the projection laser is moved into the first target position and first target orientation and the laser radiation projects onto a calculated intervention location in order to visually display the guiding axis.

20. The laser-guidance robot according to claim 19, wherein the surgery plan comprises an operation trajectory, intermediate target points and/or outlines of an operation target.

21. The laser-guidance robot according to claim 19, wherein the surgery plan comprises an operation trajectory, and the control unit sets the target orientation of the projection laser in such a way that the guiding axis projected via the laser radiation corresponds to the operation trajectory.

22. The laser-guidance robot according to claim 19, wherein the surgery plan comprises an intervention axis, and the control unit sets the target orientation of the projection laser in such a way that the guiding axis corresponds to the intervention axis.

23. The laser-guidance robot according to claim 19, wherein the surgery plan comprises at least one intervention location, an intervention axis and/or an intervention angle.

24. The laser-guidance robot according to claim 16, wherein the control unit positions and orients the projection laser relative to a predefined intervention location via the robot arm in such a way that a longitudinal axis of the laser radiation and thus the guiding axis is at a predetermined angle to the predefined intervention location.

25. The laser-guidance robot according to claim 16, wherein the laser radiation emitted by the projection laser is adapted in such a way that the laser radiation visually indicates a focal point at a predetermined intervention depth.

26. The laser-guidance robot according to claim 25, wherein the laser radiation projected by the projection laser forms a double cone having a common tip, with a focal point for a visual representation of the predetermined intervention depth at the common tip so that rings or oval shapes are formed in an intervention region above the predetermined intervention depth, the rings or oval shapes converging at the focal point at the predetermined intervention depth, and diverging below the predetermined intervention depth.

27. The laser-guidance robot according to claim 25, wherein the projection laser emits at least two laser beams that have an angle to each other and intersect each other, wherein an intersection point of the at least two laser beams indicates the predetermined intervention depth.

28. The laser-guidance robot according to claim 16, wherein the tracking system comprises an optical navigation system with a navigation camera and at least one optical tracker arranged at least on the robot head so that a position and orientation in space are precisely detected by the navigation camera to move the projection laser to the target position and target orientation relative to the intervention region.

29. The laser-guidance robot according to claim 16, wherein a position and orientation of the robot head and thus a pose of the projection laser are detected via servomotors of the laser-guidance robot.

30. The laser-guidance robot according to claim 16, wherein an orientation of the projection laser relative to the robot head is adjustable via at least one bearing.

31. The laser-guidance robot according to claim 30, wherein the orientation of the projection laser relative to the robot head is actively adjustable to a predetermined orientation relative to the robot head via a servomotor.

32. The laser-guidance robot according to claim 16, wherein preoperative images of the patient are stored in a memory unit, in particular CT images and/or MRI images, and based on a registered patient the control unit calculates an intervention axis included in the preoperative images and the projection laser is moved into this target position and target orientation, so that the guiding axis corresponds to the intervention axis.

33. A laser-guidance robot system comprising:
the laser-guidance robot according to claim 16; and
a medical instrument or medical product,
wherein the laser-guidance robot projects the guiding axis onto an intervention region via the projection laser and the medical instrument or product is alignable with a longitudinal axis along the guiding axis.

34. A projecting method for visually projecting a guiding axis onto an intervention region of a patient with a projection laser arranged on a robot head of a robot arm, the projecting method comprising the steps of:
detecting a position and/or an orientation of the projection laser in space by a tracking system;
calculating an intervention axis;
calculating a target position and target orientation of the projection laser by a control unit;
moving the robot head with the projection laser to the target position and target orientation by the robot arm; and
projecting laser radiation in the target orientation by the projection laser, so that a guiding axis projected by the projection laser indicates the intervention axis.

35. A computer-readable storage medium comprising instructions which, when executed by a computer, cause the computer to perform the projecting method according to claim 34.

36. A computer program comprising instructions which, when executed by a computer, cause the computer to perform the projecting method according to claim 34.
